(19) United States
(12) Patent Application Publication
(10) Pub. No.: US 2014/0127297 A1
(43) Pub. Date: May 8, 2014

(54) NUTRACEUTICAL COMPOSITION FOR LIMITING THE ABSORPTION OF DIETARY LIPIDS AND FOR INDUCING WEIGHT LOSS, COMPRISING, AS ACTIVE AGENT, AT LEAST ONE EXTRACT OF CARROT

(71) Applicant: Laurent Didden, Allauch (FR)

(72) Inventor: Laurent Didden, Allauch (FR)

(21) Appl. No.: 14/003,794

(22) PCT Filed: Mar. 7, 2012
(86) PCT No.: PCT/FR2012/050472

§ 371 Date: Oct. 19, 2013

(30) Foreign Application Priority Data
Mar. 8, 2011 (FR) 1151890

(51) Int. Cl. A61K 36/23; A61K 36/73; A23L 1/30; A61K 36/03

(52) U.S. Cl. CPC A61K 36/23; A61K 36/03; A61K 36/73; A23L 1/3002
USPC 424/474; 424/195.17; 424/773; 424/765

(57) ABSTRACT

The invention relates to a nutraceutical composition for limiting the absorption of dietary lipids and for inducing weight loss in a human or animal mammal, characterized in that it comprises as active agent at least one extract of carrot, at least half of which is fibres.

### TECHNICAL FIELD OF THE INVENTION

This invention has for object a nutraceutical composition for limiting the absorption of dietary lipids and for inducing weight loss comprising as active agent at least one extract of carrot.

The invention also relates to the use of such a composition as a dietary supplement or for enriching a drink or a food product.

The invention further relates to a non-therapeutic method for limiting the absorption of dietary lipids and for inducing weight loss.

### PRIOR ART

Overweight is defined as an abnormal or excessive accumulation of fats in the adipose tissue. Overweight is a major risk factor for a certain number of chronic diseases, including diabetes, cardiovascular diseases, cancer.

The main indicator for measuring overweight and obesity is the body mass index (BMI). The latter corresponds to the body weight in kilograms divided by the square of the waist in metres of a person (unit=kg/m²). In adults, a BMI between 18.5 and 24.9 is normal. A BMI between 25 and 29.9 corresponds to overweight and a BMI of 30 and more indicates obesity. These thresholds are used as points of reference for an individual evaluation, but it is evidenced that the risk of chronic diseases increases progressively above a EMI of about 22.

Due to the fact that it is difficult to restrain oneself from a dietary standpoint and to fight against undesirable calorie intake, in particular lipids, it has been shown to be desirable to be able to have a nutraceutical composition and a dietary supplement integrating such a composition that benefit people who are looking to lose weight in order to improve their health or their physical well-being, in particular overweight people who do not have any medical disorders associated with being overweight or obesity, particularly in people having a BMI index of between about 25 and about 30. A nutraceutical composition and a dietary supplement integrating such a composition based on natural products that make it possible to act on the one hand on the lipids of the alimentary bolus and on the other hand on the feeling of hunger in order to limit calorie intake are also particularly desirable in order to assist overweight people in returning to a healthy weight and people who are concerned about their figure in preventing weight gain while still allowing them to continue to have a balanced diet without have to place restrictions on themselves. It would also be desirable for this composition to not have any unpleasant side effects and that it comply with the directives in effect on dietary supplements.

Prior art knows a certain number of proposals aiming to assist overweight people in losing body weight. Application WO2000044235 (AJANTA PHARMA Limited) can be mentioned which proposes a carrot product poor in lipids comprising 20-50% by weight in carrot fibre, with the content in insoluble fibre being 15-40% by weight, and the content in soluble fibre being 5-15%. This carrot product further comprises 10-55% by weight in carbohydrates, 0.02-1% by weight in carotenoids and vitamins and 5-10% by weight in trace elements. Such a carrot product can be orally administered, in particular in order to prevent and treat diseases such as obesity, and eye diseases. Such a proposition does not appear to be of a nature to respond positively to the objectives mentioned hereinabove due to the fact that it aims for a treatment based solely on the satiety aspect. This carrot product is furthermore rich in hydrates of carbon and makes it possible to induce only a modest weight loss in obese people.

Patent FR2894777 (Laboratoires FORTE PHARMA-MC) should also be mentioned which proposes a nutraceutical composition with a cocoa fibre and orange fibre base. Such a nutraceutical composition aims solely to capture dietary fat. Furthermore, it is not demonstrated that this nutraceutical composition makes it possible to induce satisfactory loss of body weight, in particular of body fat.

It is also known through the patent US2001/012534 (BIYANI MILIND KESHARLAL), a carrot product comprising 20-50% by weight in carrot fibre, with the content in insoluble fibre being 15-40% by weight, and the content in soluble fibre being 5-10%, 10-55% by weight in hydrates of carbon, 0.02-1% by weight in carotenoids and vitamins and 5-10% by weight in minerals and trace elements. Such a carrot product can be administered orally, in particular to prevent and treat diseases such as obesity, and eye diseases. The oral administration of such a product makes it possible to improve the feeling of satiety and to induce weight loss limited to 2 to 3 kg in obese people over a period of 2 months (see example 18). This low weight loss likely comes from the fact that the lipids supplied by the diet are not effectively taken up, but on the contrary massively absorbed and stored in the organism.

In light of this situation, an objective of the invention is to propose a nutraceutical composition which makes it possible to effectively limit the absorption of dietary lipids and to induce significant weight loss in mammals, human or animal.

Another objective of the invention is to provide a composition making it possible to effectively capture the lipids supplied by the diet, as such limiting their absorption and their storage in the organism, and to induce satisfactory weight loss in a mammal, human or animal, in particular in a human subject having a BMI between about 25 and about 30.

Another objective of the invention is to propose a nutraceutical composition of which the active agent is particularly simple to obtain.

### DIVULGATION OF THE INVENTION

The solution proposed by the invention is a nutraceutical composition for limiting the absorption of dietary lipids and for inducing weight loss in a mammal, human or animal. This composition comprises as active agent at least one extract of carrot having a total fibre content between 50% and 99% by weight based on the total dry weight of the extract of carrot, preferably between 60% and 95% by weight based on the total dry weight of the extract of carrot, and additional dietary fibre constituted mostly of insoluble fibre and obtained from apple and/or oat, with the content in said extract of carrot being at least 20% p/p_{composition} and the content in said additional dietary fibre being less than or equal to 10% p/p_{composition}.

The applicant showed that an extract of carrot makes it possible to effectively capture the lipids supplied by the diet, as such limiting their absorption and their storage in the organism and as such contributing to the fight against undesirable weight gain and to inducing satisfactory weight loss in a mammal, human or animal, in particular in a human subject having a BMI between about 25 and about 30.

These results are especially surprising in that there is no data, more particularly, in vivo, known to date that demonstrates the effectiveness of carrot fibre in limiting the absorption of dietary lipids and in inducing a satisfactory loss of body weight.

As such, according to a first aspect, the invention has for object a composition for fighting against the absorption of dietary lipids and for inducing weight loss in a mammal, human or animal, comprising as active agent at least one extract of carrot.

The extract of carrot is preferably present in the composition for at least 20% p/p_{composition}, preferably for at least 40% p/p_{composition}.

Advantageously, the extract of carrot is mostly constituted of insoluble fibre. The weight ratio between insoluble fibre and soluble fibre is between 1.02 and 20, preferably between 1.5 and 10, or even better about 6.

Preferably, the composition according to the invention further comprises from 2% to 30% p/p_{composition} of a plant extract containing a lipase and/or α-amylase and/or α-D-glucosidase inhibitor.

The plant extract containing a lipase and/or α-amylase and/or α-D-glucosidase inhibitor is advantageously selected from the group consisting of: extract of brown algae, extract of white bean, extract of Cassia nomame, extract of the mushroom Phellinus linteus, extract of green tea, extract of black tea, an extract of kiwi, extract of lichen Umbilicaria esculenta, extract of the Vietnamese plant Cleistocalyx operculatus, extract of bitter melon Mormodica charantia, extract of guava (leaves) or others.

Preferentially, the plant extract containing a lipase and/or α-amylase and/or α-D-glucosidase inhibitor is an extract of brown algae Ascophyllum nodosum.

The composition can also contain a quantity less than or equal to 10% p/p_{composition} of additional dietary fibre, in particular in order to enrich said composition with insoluble fibre for the purpose of reinforcing its action against the absorption of total calories, in particular of dietary lipids for the purpose of inducing weight loss, but also in order to standardise the functions of the colon, regulate the intestinal transit and fight against the tendency to constipation.

Preferably, this additional dietary fibre is constituted mostly of insoluble fibre and can be obtained from plants, fruits, vegetables, cereals, roots or mixtures of them.

Advantageously, the additional dietary fibre is obtained from apple and/or oat.

The composition can furthermore include a quantity less than or equal to 5% p/p_{composition} of a dry extract of apple, in particular in order to enrich the composition with polyphenolic compounds, of which phloridzine, which are known for modulating postprandial glycaemia and in order to regulate the absorption of cholesterol, and this with the purpose of reinforcing the action of the composition aiming to limit the absorption of dietary lipids and reducing the storage of fat.

The composition can furthermore include a quantity less than or equal to 10% p/p_{composition}, in particular in order to contribute to moderating the appetite and to regulating the intestinal transit. Preferably apple pectin is present in the composition at a content between 4% and 9% p/p_{composition}.

Preferably, the composition comprises:

from 17% to 20% p/p_{composition} of extract of brown algae, preferably about 17.3% p/p_{composition},

from 45% to 55% p/p_{composition} of extract of carrot, preferably about 48% p/p_{composition},

from 3% to 4% p/p_{composition} of apple fibre, preferably about 3.5% p/p_{composition},

from 3% to 4% p/p_{composition} of oat fibre, preferably about 3.5% p/pcomposition,

from 2% to 3% p/p_{composition} of extract of apple, preferably about 2.5% p/p_{composition}, and,

excipients qsp 100%

The composition is preferably formulated for oral administration, and has for example, the form of powders, scored or unscored tablets, film-coated or not, granules, capsules, gel capsules.

Another aspect of the invention relates to the use of the aforementioned composition as a dietary supplement, or to enrich a drink or a food product.

Yet another aspect of the invention relates to a non-therapeutic method for limiting the absorption of calories, in particular of dietary lipids and for inducing weight loss, with the nutraceutical composition being administered to a mammal, human or animal, at a daily dose of about 300 mg/meal to about 1600 mg/meal.

Other purposes and advantages of this invention shall appear in the following description which is provided solely for the purposes of information and which does not intend to limit it.

### EMBODIMENTS OF THE INVENTION

The nutraceutical composition object of the invention can be used in a mammal, human or animal, for limiting the absorption of dietary lipids and for inducing weight loss. It is used in a subject who desires to lose a portion of body weight and of body fat in order to improve his health or desires to slim for his physical well-being. And in particular, but not exclusively, a subject having a BMI between about 25 and about 30.

In accordance with the invention, extract of carrot is used as active agent for limiting the absorption of dietary lipids and for inducing weight loss. In the context of the invention, “weight loss” means the loss of a portion of the total weight in a mammal, human or animal, in particular the loss of a portion of the body fat.

The carrot, or Daucus carota var. sativus, belongs to the family of the Apiaceae. In practice, a dry extract of carrot is used which is obtained from mashed carrots (Daucus carota) of which the juice is totally separated via conventional extracting and separating techniques known to those skilled in the art. Such an extract contains soluble fibre (pectins) and insoluble fibre (cellulose and hemicellulose), minerals and a low fraction of lipids and proteins.

The composition of the invention comprises at least 20% p/p_{composition}, preferably at least 40% p/p_{composition} of the extract of carrot.

The extract of carrot utilised preferably has a content in total fibre (soluble and insoluble fibre) between 50% and 99%, advantageously between 60% and 95%, preferably about 92%, by weight based on the total dry weight of the extract of carrot. The content in total fibre is determined according to official method No. 56-20 amended of the AACC (American Association of Cereal Chemists). The extract of carrot used is mostly constituted of insoluble fibre, with the weight ratio between insoluble fibre and soluble fibre being between 1.02 and 20, advantageously between 1.5 and 10, preferably about 6.

The extract of carrot is advantageously used in the form of fine powder of which about 20% of the particles have a diameter less than 40 μm and about 98% of the particles have a diameter less than 100 μm. An extract of carrot marketed by the company ID FOOD under the name ID 809® for example is used.

Such an extract of carrot has a fat retaining capacity between 300% and 500% and a high absorption capacity for water between 1500 and 3000%, for example of about 1800%. The fat retaining capacity and the absorption capacity are determined by the method No. 56-20 amended of the AACC. The extract of carrot therefore provides the nutraceutical composition with the faculty of effectively capturing the dietary lipids along their travel in the stomach and the intestine optimally limiting as such the caloric intake of the ingested foods.

The nutraceutical composition can furthermore include, a plant extract containing a lipase and/or α-amylase and/or α-D-glucosidase inhibitor, in particular in order to limit the assimilation of the fats and/or of the sugars of the alimentary bolus and decrease the accumulation of fats in the organism with the purpose of fighting against the global intake of calories and of inducing weight loss. In order to optimise this effect, the content of this plant extract is between 2% and 30% p/p_{composition}.

In the context of this invention,

“lipase inhibitor” means a compound able to inhibit the action of the gastric and pancreatic lipases responsible for the metabolism and the absorption of the fats.

“α-amylase inhibitor” means a compound able to inhibit the action of the α-amylase which is involved in the digestion of the carbohydrates, for example of the α-amylase secreted by the salivary glands and by the pancreas.

“α-D-glucosidase inhibitor” means a compound able to inhibit the action of the α-D-glucosidase that hydrolyses the disaccharides and/or the polysaccharides linked by an α-(1.4) bond in order to release for example the glucose.

By way of example of a plant extract containing a lipase and/or α-amylase and/or α-D-glucosidase inhibitor the following for example can be mentioned: extract of brown algae, extract of white bean, extract of Cassia nomame, extract of the mushroom Phellinus linteus, extract of green tea, extract of black tea, extract of kiwi, extract of lichen Umbilicaria esculenta, extract of the Vietnamese plant Cleistocalyx operculatus, extract of bitter melon Mormodica charantia, extract of guava (leaves) or others.

The applicant surprisingly observed that an extract of brown algae Ascophyllum nodosum potentiates the effect of the extract of carrot.

Ascophyllum nodosum or Ascophylle noueuse is a marine algae classed in the family of the Fucaceae. The extract of brown algae Ascophyllum nodosum used in this invention can be prepared by any extraction and purification method known to those skilled in the art. It can also be obtained from Laboratoires Bio Serae® SAS under the trade name ID-alG®.

In background art, it was discovered that associating the extract of carrot and the extract of brown algae Ascophyllum nodosum has a complementary and synergistic effect for acting on the absorption of dietary lipids and carbohydrates and for inducing a significant weight loss. It has in particular been demonstrated that this new association allows for an action on three levels, i.e. capturing of dietary lipids, an inhibition of the digestive enzymes involved in the assimilation of the fats and of the sugars and an induction of the feeling of satiety.

The nutraceutical composition can furthermore include additional dietary fibre, in particular for enriching said composition with insoluble fibre for the purpose of reinforcing its action against the absorption of total calories, in particular of dietary lipids, for the purpose of inducing weight loss, but also for standardising the functions of the colon, regulating the intestinal transit and fighting against the tendency to constipation. The additional dietary fibre used are constituted mostly of insoluble fibre and are present in the composition according to the invention at a content less than 10% p/p_{composition}. Such additional dietary fibre, can for example be obtained from: plants such as green tea, black tea, rhubarb stalks, cocoa, or others; legumes such as lentils, white or black beans, beans, chickpeas, or others; fruits such as citrus, raspberries, apples or others; vegetables such as nopal, celery, green beans, carrots, tomatoes (skins), potatoes (skins) or others; cereals such as wheat, oat, corn, linseed or others; or, mixtures of them.

It is preferred however to use additional dietary fibre obtained from apple and/or oat, in particular for the intake of the composition in insoluble fibre and for optimising the action of the composition aimed at limiting the absorption of total calories, in particular of dietary lipids and at inducing a loss in body weight, but also for standardising the functions of the colon, regulating intestinal transit and fighting against the tendency to constipation.

The apple fibre used comes from apple (Pyrus malus) after separation of the juice by conventional extraction and separation techniques known to those skilled in the art. They have a total fibre content (soluble and insoluble fibre) of about 63% by weight based on the total weight of the dry matter. The insoluble fibre and the soluble fibre represent respectively about 67% and 33% by weight based on the total weight of the dry matter. The apple fibre is advantageously used in this invention in the form of fine powder of which about 98% of the particles have a diameter less than 315 μm and 60% of the particles have a diameter less than 150 μm. The apple fibre can be present in the nutraceutical composition at a content less than or equal to 10% p/p_{composition}, advantageously at a content ranging from 3 to 4% p/p_{composition}, preferably at a content of about 3.5% p/p_{composition}. They are for example sold under the trade name ID 75® by the Company ID FOOD®.

The oat fibre used can be obtained according to any conventional extraction and purification method known to those skilled in the art using oat hulls (Avena sativa). Preferably, the oat fibre have a total fibre content (soluble and insoluble fibre) of about 96% by weight based on the total weight of the dry matter and a water content less than 8%, by weight based on the total weight of the dry matter. The insoluble fibre and the soluble fibre represent respectively about 93% and 3% by weight based on the total weight of the dry matter. The oat fibre is used in this invention in the form of fine powder of which at most 14% of the particles have a diameter greater than 32 μm and at most 0.5% of the particles have a diameter greater than 71 μm. The oat fibre can be present in the nutraceutical composition at a content less than or equal to 10% p/p_{composition}, advantageously at a content ranging from 3 to 4% p/p_{composition}, preferably at a content of about 3.5 p/p_{composition}. They are for example sold under the trade name ID 914® and can be obtained from the Company ID FOOD®.

An extract of apple can furthermore be used to enrich the composition with polyphenolic compounds, of which phloridzine, which make it possible to modulate the postprandial glycaemia and to regulate the absorption of the cholesterol, with the purpose of reducing the storage of fat. This extract of apple is preferably present in the composition at a content less than or equal to 5% p/p_{composition}. Such an extract of apple is for example Nutricible® Apple PHZ which is manufactured and marketed in France by the company LabAttitude®.

The composition can also contain apple pectin, in particular to contribute in managing dietary intake, in particular in moderating the appetite, in slowing down the absorption of lipids and carbohydrates. This apple pectin acts in synergy with the extract of carrot to induce weight loss and favour intestinal transit. The apple pectin used is a highly methylated pectin having preferably a degree of esterification between 56-63%. Such an apple pectin is mainly comprised of partial methyl esters of polygalacturonic acid and of their ammonia, sodium, potassium, and/or calcium salts. The apple pectin is present in the composition at a content less than or equal to 10% p/p_{composition}, preferably between 4% and 9% p/p_{composition}. It is for example sold under the trademark AF 501® by the German Company Herbstreith-Fox®.

Moreover, the composition of the invention can furthermore contain substances able to facilitate the destruction of the reactive oxygen species such as vitamins E and C, carotenoids, mineral micronutrients such as selenium and zinc.

Advantageously, the composition of the invention can furthermore include any suitable excipient, acceptable from a nutraceutical standpoint, and known to those skilled in the art. In terms of excipients, dicalcium phosphate, calcium alginate, magnesium carbonate, talc, magnesium stearate, silicon dioxide, calcium chloride, or others can for example be mentioned.

A preferred composition of the invention comprises:

from 17% to 20% p/p_{composition} of extract of brown algae, preferably about 17.3% p/p_{composition},

from 45% to 55% p/p_{composition} of extract of carrot, preferably about 48% p/p_{composition},

from 3% to 4% p/p_{composition} of apple fibre, preferably about 3.5% p/p_{composition},

from 3% to 4% p/p_{composition} of oat fibre, preferably about 3.5% p/p_{composition},

from 2% to 3% p/p_{composition} of extract of apple, preferably about 2.5% p/p_{composition}, and

excipients qsp 100%

The nutraceutical composition can be administered orally, for example, in the form of scored or unscored tablets, film-coated or not, granules, capsules, gel capsules, or in the form of loose powder packaged preferably in single sachets, or compressed powder.

For a non-therapeutic treatment in the fight against the absorption of lipids and a programme for weight loss (subject having a BMI less than or equal to 30), the composition object of the invention can be administered to a mammal, human or animal, at a daily dose of about 300 mg/meal to about 1600 mg/meal. In practice, a dosage adapted for limiting the absorption of dietary lipids and for inducing weight loss is preferably from 3 to 6 gel capsules per day. By way of example, the composition can be taken:

during a continuous programme called “weight loss” of a duration ranging from two to four weeks, and at a rate of 3 gel capsules at noon and 3 gel capsules in the evening during meals with a glass of water, or,

occasionally, at a rate of 3 gel capsules during at least one copious meal at noon and/or in the evening, with the purpose of reducing any dietary excesses.

The continuous “weight loss” programme can possibly be extended until the desired weight loss is obtained, given that the dietary supplement according to the invention is comprised of natural products which do not have any unpleasant side effects and which are of no danger to health.

The composition object of the invention can also be used during a therapeutic treatment in fighting against the absorption of lipids and a programme for weight loss, in particular in subjects having a BMI greater than 30. The dosage is then defined according to the case at hand.

The composition when it is in the form of powder can be incorporated into all forms of enriched food products, for example food bars, savoury and/or sweet biscuits or others. It can also be dispersed in water, in drinks such as soft drinks or juice or in dairy products or derivatives of soya, or others.

The composition according to the invention can in particular be used as a dietary supplement, presented in any form that is compatible with an oral absorption in one or more daily takings. This dietary supplement can in particular have the form of gel capsules, capsules, tablets, film-coated or not, scored or unscored, granules, or in the form of loose powder, preferably packaged in single sachets, or compressed powder. Dietary supplement means an edible set intended to supplement the current diet of a human or animal organism, by supplying it with nutrients or other substances having for function to overcome the insufficiencies of a poorly balanced diet. The dietary supplement, is therefore by definition a food item of which the purpose is to supplement a normal diet which constitutes a concentrated source of a nutrient or of other substances having a nutritional or physiological effect, alone or combined, marketed in the form of a dose. Dose means any of the conventional forms, i.e. gel capsule, lozenge, tablet, granules, pill, and other similar forms as well as powder sachets.

### EXAMPLES OF COMPOSITIONS OR OF DIETARY SUPPLEMENTS IN ACCORDANCE WITH THE INVENTION

### Example 1

### Composition of a Dietary Supplement Presented in the Form of Gel Capsules

A gel capsule is prepared according to the usual techniques dosed at 120 mg of extract of carrot, of oat fibre, and of apple pectin having the following composition:

| Ingredients | Weight (mg) |
|---|---|
| Extract of carrot | 90.00 |
| Oat fibre | 15.00 |
| Apple pectin | 15.00 |
| Dicalcium phosphate | 76.00 |
| Magnesium carbonate | 50.00 |
| Talc | 9.50 |
| Magnesium stearate | 4.50 |
| Silicon dioxide | 3.00 |

The various ingredients have the form of powders. They are independently weighed then incorporated into one another. This composition is designated hereinafter as “composition A”.

### Example 2

### Nutraceutical Composition Presented in the Form of Powder

A composition is prepared according to the usual techniques in the form of powder containing an extract of carrot, an extract of brown algae Ascophyllum nodosum, apple fibre, oat fibre, an extract of apple, and having the following composition:

| Ingredient | Weight (mg) |
|---|---|
| Extract of carrot | 166.67 |
| Extract of brown algae | 66.50 |
| Oat fibre | 12.50 |
| Apple fibre | 12.50 |
| Extract of apple | 8.33 |

The various ingredients have the form of powders. They are independently weighed, then incorporated into one another. This composition is designated hereinafter as “composition B”.

### Example 3

### Composition of Another Dietary Supplement Presented in the Form of Gel Capsules

A gel capsule is prepared according to the usual techniques containing an extract of carrot, an extract of brown algae Ascophyllum nodosum, apple fibre, oat fibre, an extract of apple and excipients and having the following composition:

| Ingredients | %, p/p_{composition} |
|---|---|
| Extract of carrot | 48.97 |
| Extract of brown algae | 19.12 |
| Oat fibre | 3.68 |
| Apple fibre | 3.68 |
| Extract of apple | 2.50 |
| Calcium alginate | 14.71 |
| Calcium chloride | 7.35 |

All of the ingredients are incorporated in the form of powder. They are independently weighed then incorporated into one another.

### Evaluation of the Effectiveness in Vitro of the Capture of Dietary Fats Contained in a Fatty Product, via a Control and via Composition B of the Example 2 Hereinabove

The capacity of composition B to capture the dietary fats was evaluated in vitro by using the method described in patent application FR2894777 (Forté Pharma).

The control used is the product CaloriLight® which is marketed by the company Forté Pharma®.

The tested products are chocolate, crisps and pistachios: these products were chosen because they are rich in calories, in particular in lipids and represent frequently consumed foods.

The capacity of the fibre to fix the lipids (Cg) is expressed by the following ratio:

$$Cg = \frac{M 1 - M 3}{M 2}$$

wherein,

Cg=quantity of lipids (g) captured per gram of composition B or of the control.

M1=mass of lipids introduced

M2=mass introduced of composition B or of the control

M3=mass of supernatant lipids

The results are shown in the following tables 1 to 3:

TABLE 1
results obtained for chocolate

| Sample | M1 (mg) | M2 (mg) | M3 (mg) | Average Cg |
|---|---|---|---|---|
| control | 3104.63 | 270.27 | 673.5 | 8.995 |
| Composition B | 3044.84 | 266.62 | 78.08 | 11.144 |

TABLE 2
results obtained for crisps

| Sample | M1 (mg) | M2 (mg) | M3 (mg) | Average Cg |
|---|---|---|---|---|
| control | 2997.1 | 276.14 | 1427.87 | 5.684 |
| Composition B | 2955.33 | 263.47 | 316.83 | 10.017 |

TABLE 3
results obtained for pistachios

| Sample | M1 (mg) | M2 (mg) | M3 (mg) | Average Cg |
|---|---|---|---|---|
| control | 3050.30 | 270.67 | 759.73 | 8.463 |
| Composition B | 2999.68 | 262.83 | 71.46 | 11.148 |

Composition B has a capacity for capturing dietary fats that is greater than that of the control product by about:

24% for chocolate

76% for crisps

32% for pistachios

The percentage of absorption of lipids by composition B is shown in table 4 hereinbelow. It is expressed by the following formula:

$$percentage of absorption of lipids {=}^{\frac{M 5}{M 1} \cdot 100}$$

wherein M5=mass of lipids absorbed by composition B=M1−M3

TABLE 4
percentage of absorption of lipids

| | | | | Percentage |
|---|---|---|---|---|
| | | | | of |
| | | | | absorption |
| Source of | | | | of lipids |
| lipids | M1 (mg) | M3 (mg) | M5 (mg) | (%) |
| chocolate | 3044.84 | 78.08 | 2966.76 | 97 |
| crisps | 2955.33 | 316.83 | 2638.50 | 89 |
| pistachios | 2999.68 | 71.46 | 2928.22 | 98 |

Composition B therefore makes it possible to absorb about:

97% of the lipids contained in the chocolate

89% of the lipids contained in the crisps

98% of the lipids contained in the pistachios

If it is considered that composition B corresponds to the contents of one gel capsule (266.67 mg of active ingredients), it can be deduced that taking 3 gel capsules (0.8 g of active ingredients) would make it possible to capture about:

8.92 g of lipids supplied by the chocolate, or a little more than the quantity of lipids supplied by 6 squares of chocolate, given that 100 g of chocolate contain about 28.8 g of lipids,

8.01 g of lipids supplied by the crisps, or a little more than the quantity supplied by a packet of 25 g crisps, given that 100 g of crisps contain about 36 g of lipids.

8.91 g of lipids supplied by about 16.5 g of pistachios, given that 100 g of pistachios contain about 54 g of lipids.

### Clinical Study

The effectiveness and the tolerance of composition A described in example 1, was studied on overweight volunteers. An open clinical trial was conducted on 25 male or female volunteers, aged between 18 and 65 years and responding to the criteria for pre-obesity i.e. having a Body Mass Index (BMI) greater than or equal to 25 and less than 30, and this for a duration of 15 and 30 consecutive days.

The male or female volunteers presented themselves 3 times on D0, D15 and D30. During D0 the volunteers are weighed, measured in order to check the BMI. The perimeter of the right thigh, the waist size and the hip size are also taken. At D15 and D30, another weighing and measurements of the different perimeters are then taken.

The volunteers were furthermore asked to evaluate their feeling of satiety over a given period.

The dosage is 3 gel capsules at midday and 3 gel capsules in the evening, at the beginning of the meal with half a glass of water.

Dietary advice was offered to the volunteers (maintaining a balanced diet in terms of quality and quantity) in order to control the conditions of the study.

The results of this clinical study are that 100% of the volunteers lost weight homogeneously. An average weight loss of −3.19 Kg (4.26%) was obtained over a duration of 15 days, with a maximum loss of −8.4 kg for the same duration, and an average weight loss of −6.03 kg (8.04%) was obtained over 30 days, with a maximum loss of −10.7 kg for the same duration. The difference between D30 and D0 were statistically significant (p<0.0001).

The weight loss is obtained solely with regards to body fat, since an average loss of −5.8 Kg was obtained on the body fat (significant between D0 and D30, p<0.0001), with the loss of lean body mass representing less than 0.5% of the total weight lost in 30 days. This is a total average weight loss of about 1.5 kg/week and of body fat of about 1.45 kg/week.

The waist size was measured using a perimeter tape. A perimeter decrease was observed in 100% of the volunteers. The average loss of the waist size obtained is −2.68 cm (2.86%) over 15 days and −4.81 cm (4.81%) over 30 days, with the maximum loss obtained being −10 cm in 30 days. This is an average loss in waist size of at least 1.2 cm/week.

The weight loss is obviously passed on to the body mass index: the average loss was −2.20 kg/m² (7.93%), with a maximum loss of −3.9 kg/m² over 30 days. The difference between D30 and D0 is significant (p<0.0001).

The results show that 100% of the volunteers lost on average about 1.5 kg in total weight in the first week and lost substantially in volume, in particular around the waist where the result is maximum up to −10 cm in 30 days. The weight loss was obtained solely with regards to body fat.

Moreover, the composition evaluated did not have any unpleasant side effects.

Furthermore, more than 90% of the volunteers confirmed that composition A provided them with a very strong feeling of satiety as well as a general feeling of well-being.

1. A nutraceutical composition for limiting the absorption of dietary lipids and for inducing weight loss in a mammal, human or animal, characterised in that it comprises as active agent an extract of carrot having a content in total fibre between 50% and 99% by weight based on the total dry weight of the extract of carrot, and additional dietary fibre constituted mostly of insoluble fibre and obtained from apple and/or oat, with the content in said extract of carrot being at least 20% p/p_{composition} and the content in said additional dietary fibre being less than or equal to 10% p/p_{composition}.

2. A nutraceutical composition according to claim 1, characterised in that the extract of carrot contains mostly insoluble fibre.

3. A nutraceutical composition according to claim 2, characterised in that the weight ratio between insoluble fibre and soluble fibre is between 1.02 and 20.

4. A nutraceutical composition according to claim 1, characterised in that it further comprises from 2% to 30% p/p_{composition} of a plant extract containing a lipase and/or α-amylase and/or α-D-glucosidase inhibitor.

5. A nutraceutical composition according to claim 1, characterised in that the plant extract containing a lipase and/or α-amylase and/or α-D-glucosidase inhibitor is selected from the group consisting of an extract of brown algae, extract of white bean, extract of Cassia Nomame, extract of the mushroom Phellinus Linteus, extract of green tea, extract of black tea, an extract of kiwi, extract of lichen Umbilicaria esculenta, extract of the Vietnamese plant Cleistocalyx operculatus, extract of bitter melon Mormodica charantia, extract of guava.

6. A nutraceutical composition according to claim 1, characterised in that the plant extract containing a lipase and/or α-amylase and/or α-D-glucosidase inhibitor is an extract of brown algae Ascophyllum nodosum.

7. A nutraceutical composition according to claim 1, characterised in that it further comprises a dry extract of apple at a content less than or equal to 5% p/p_{composition}.

8. A nutraceutical composition according to claim 1, characterised in that it further comprises the apple pectin at a content less than or equal to 10% p/p_{composition}.

9. A nutraceutical composition as claimed in claim 1, characterised in that it comprises:
from 17% to 20% p/p_{composition} of extract of brown algae,
from 45% to 55% p/p_{composition} of extract of carrot,
from 3% to 4% p/p_{composition} of apple fibre,
from 3% to 4% p/p_{composition} of oat fibre,
from 2% to 3% p/p_{composition} of dry extract of apple, and,
excipients qsp 100%.

10. A nutraceutical composition according to claim 1, characterised in that it has the form of powder, of scored or unscored tablets, film-coated or not, of capsules or of gel capsules.

11. A nutraceutical composition according to claim 1, characterised in that it has a form that is suitable for oral administration.

12. Use of the nutraceutical composition according to claim 1, as a dietary supplement or for enriching a drink or a food product.

13. Non-therapeutic method for limiting the absorption of calories, in particular of dietary lipids and for inducing weight loss, characterised in that the nutraceutical composition according to claim 1 is administered to a mammal, human or animal, at a rate of a daily dose of about 300 mg/meal to about 1600 mg/meal.

14. A nutraceutical composition according to claim 1 characterised in that the extract of carrot has a content in total fibre between 60% and 95% by weight based on the total dry weight of the extract of carrot.

15. A nutraceutical composition according to claim 1 characterised in that the weight ratio between insoluble fibre and soluble fibre is between 1.5 and 10.

16. A nutraceutical composition according to claim 1 characterised in that it comprises:
about 17.3% p/p_{composition} of extract of brown algae,
about 48% p/p_{composition} of extract of carrot,
about 3.5% p/p_{composition} of apple fibre,
about 3.5% p/p_{composition} of oat fibre,
about 2.5% p/p_{composition} of dry extract of apple.
